(19) United States
(12) Plant Patent Application Publication
(10) Pub. No.: US 2018/0084703 P1
(43) Pub. Date: Mar. 22, 2018

(54) Lobelia variety name 'Almanda Blue'

(50) ;

(71) Applicant: WIRRAPUNGA PTY LTD, North Adelaide (AU)

(72) Inventor: John Wamsley, Aldgate (AU)

(21) Appl. No.: 15/330,447

(22) Filed: Sep. 22, 2016

(51) Int. Cl. A01H 5/02

(52) U.S. Cl. PLT/451

(57) ABSTRACT

A new and distinct variety of Lobelia pedunculata named ‘ALMANDA BLUE’, particularly characterized by prolific, mid-blue, irregular flowers on long peduncles, a dense weeping habit; and quick growth. Other desirable characteristics include female only flowers, upright flower peduncles, fast propagation from runners, a short timeframe to flowering from propagation, and tolerance to damp conditions. Plants prefer full sun to part shade and temperatures ranging from 0° C. to about 35° C., but will withstand minor frosts and heat extremes to about 40° C. for short periods.

Latin name of the genus and species of the plant claimed: Lobelia pedunculata.

Variety denomination: ‘ALMANDA BLUE’.

### BACKGROUND OF THE INVENTION

The disclosure provides a new and distinct variety of Lobelia, botanically known as Lobelia pedunculata, and hereinafter referred to by the variety name ‘ALMANDA BLUE’.

The new variety arose from a spontaneous mutation of Lobelia pedunculata in John Wamsley's bushland garden. The new variety of Lobelia was selected for development on basis of vigour, plant habit (weeping), and prolific flowering.

The new variety consists of one female plant. It was then propagated via vegetative cuttings and has been asexually reproduced repeatedly by vegetative cuttings for four cycles of selection and six generations in its present form.

Asexual reproduction of the new Lobelia plant has shown that the unique features of this new Lobelia plant are stable and reproduced true to type in successive generations of asexual reproduction.

### SUMMARY OF THE INVENTION

The following traits have been repeatedly observed and are determined to be the most outstanding and distinguishing characteristics of ‘ALMANDA BLUE’, a new variety of Lobelia, when grown under normal horticultural practices:

1. Prolific, mid-blue, irregular flowers on long peduncles;

2. Dense weeping habit; and

3. Quick growth.

Other desirable characteristics include female only flowers, upright flower peduncles, fast propagation from runners, a short timeframe to flowering from propagation, and tolerance to damp conditions. Plants prefer full sun to part shade and temperatures ranging from 0 to 35° C., but will withstand minor frosts and heat extremes to about 40° C. for short periods.

‘ALMANDA BLUE’ differs from the parent plant Lobelia pedunculata (unpatented) in that ‘ALMANDA BLUE’ has irregular flower shape, weeping attitude of stem, absent or very sparse pubescence and mid blue flower color, whereas Lobelia pedunculata has salverform flower shape, more upright stem attitude, dense pubescence and pale blue flower color.

‘ALMANDA BLUE’ differs from Lobelia with the closest flower shape Lobelia anceps (unpatented) in that ‘ALMANDA BLUE’ is prostrate, has broad ovate leaf shape, broad leaf width and short leaf length, whereas Lobelia anceps is tall, has cuneate leaf shape, narrow leaf width, and long leaf length.

### BRIEF DESCRIPTION OF THE DRAWING

The accompanying photographs (drawings) illustrate the overall appearance of the new Lobelia pedunculata ‘ALMANDA BLUE’ and show overall plant habit, including blooms and foliage of the plant in full color. The photographs show the colors of ‘ALMANDA BLUE’, as true as is reasonably possible, with conventional photographic procedures and digital reproduction. Colors in the photographs may differ slightly from the color values cited in the detailed botanical description, which accurately describe the color of ‘ALMANDA BLUE’.

FIG. 1 shows a typical 4-month old plant of ‘ALMANDA BLUE’ in the spring of 2015. The plant was grown in a 20 centimeter pot in Aldgate, South Australia (altitude 304 m, −35.01S 138.73E) in a polycarbonate greenhouse, with a temperature range of about 18-25° C. ±3° C., under natural daylight and day length, under normal horticultural practices.

FIG. 2 shows a close up view of the flowers, 8 weeks after propagation, in the middle of March (early Autumn). Plants were grown at the Waite Campus (Urrbrae South Australia, altitude 110 m, 34.96S 138.63E) in a polycarbonate greenhouse, with a temperature range of about 18-25° C. ±3° C., under natural daylight and day length, and about 30% shade produced by greenhouse blinds, under normal horticultural practices.

### DETAILED BOTANICAL DESCRIPTION

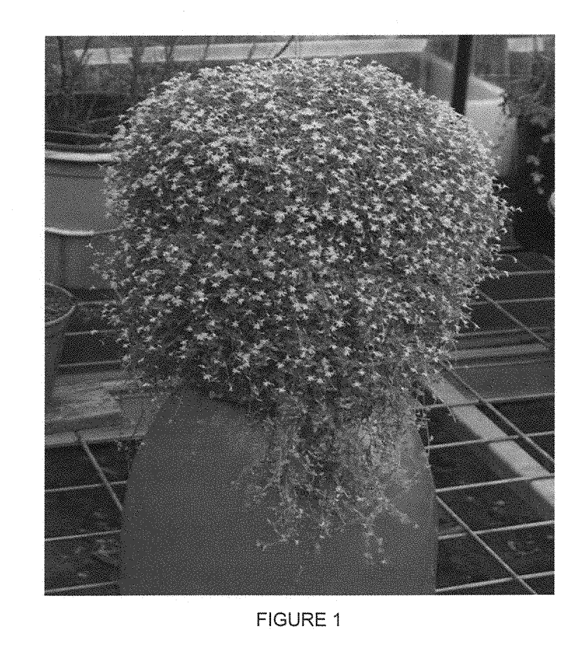

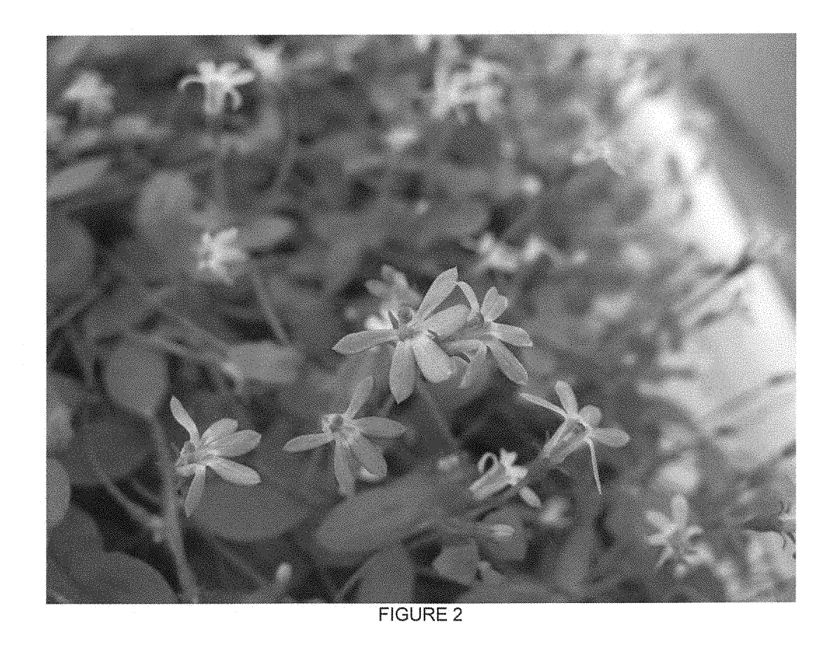

Plants used in the aforementioned photographs and in the following description were grown inside under greenhouse conditions and cultural practices which approximate those generally used in South Australia. More specifically, plants in FIG. 1 were grown in a polycarbonate greenhouse in Aldgate, South Australia (altitude 304 m, −35.01S 138.73E) with a temperature range of about 18-25° C. ±3° C., under natural daylight and day length, under normal horticultural practices. Plants in FIG. 2 were grown in a polycarbonate greenhouse at the Waite Campus, Urrbrae South Australia (altitude 110 m, 34.96S 138.63E) with a temperature range of about 18-25° C. ±3° C., under natural daylight and day length, and about 30% shade produced by greenhouse blinds, under normal horticultural practices. Measurements and numerical values represent averages for typical flowering plants.

The following detailed description sets forth the distinctive characteristics of ‘ALMANDA BLUE’. The data which define these characteristics were collected from asexual reproductions carried out in Aldgate, South Australia. The plant history was taken in the spring of 2015 on about 4-month old plants grown in 20 centimetre pots in a plastic greenhouse. This description is in accordance with terminology of the International Union for the Protection of New Varieties of Plants (UPOV).

Color designations, color descriptions, and other phenotypical descriptions may deviate from the stated values and descriptions depending upon variations in environmental, seasonal, climatic, and cultural conditions. Color readings were taken under natural light. Color references are primarily to the Colour Charts of The Royal Horticultural Society of London (R.H.S.), i.e., R.H.S. Colour Charts, Fifth Edition (2007). Color readings were taken under conditions of natural light.

All of the plants of ‘ALMANDA BLUE’, insofar as they have been observed, have been consistent in the characteristics described below.
Classification:
   Botanical.—Lobelia pedunculata.
   Common name.—Matted Pratia.
   Designation.—‘ALMANDA BLUE’.
Parentage:
   Female, or seed parent.—Lobelia pedunculata, unpatented.
   Male, or pollen parent.—None.
Propagation:
   Type.—Runners (above ground stolon).
   Time to initiate roots.—already rooted when planted.
   Time to produce a young plant.—About 6 weeks.
   Root description.—Fine, white roots, freely branching and dense.
   Time to produce a finished flowering plant.—About 12 weeks.
   Outdoor plant performance.—Very good.
Plant description:
   Form.—Prostrate.
   Plant height, measured from the top of the soil, including any flowers.—About 12.0 cm.
   Plant width, horizontal plant diameter, including any flowers.—About 44.0 cm.
   Growth and branching habit.—Weeping.
   Lateral branching description.—There are two distinct types of branches. There are runners and there are flowering branches. If a rooted runner cutting is planted in a pot, then the first growth will be a series of runners. These are prostrate and grow out from the cutting. These runners will root at the nodes if they are in contact with damp soil or water. They will grow over the edge of the pot and hang down completely covering the pot. The length of these runners, in some aspects, is up to about 60 cm long. The inter-nodal length, in various aspects, ranges from about 0.5 cm to about 3 cm. The diameter is about 0.1 cm. There is a leaf at each node. Lateral branches then grow from each node. The lateral branch can be another runner or a flowering branch. Flowering branches are very different to runners. In flowering branches, flowering stems curl upwards and flower from the leaf axils. The result is that the plant completely covers the pot (FIG. 1). Flowers then grow out on long peduncles and give the effect of a blue aura or halo over the green background of the plant (FIG. 1).
Leaf description:
   Arrangement.—Alternate.
   Length.—About 0.6 to 1.0 cm.
   Width.—About 0.5 to 0.7 cm.
   Shape.—Broad ovate.
   Margin.—Few widely spaced teeth, each somewhat thickened.
   Apex.—Acute.
   Base.—Acute.
   Texture, both upper and lower surfaces.—Smooth.
   Venation pattern.—Pinnate venation color: RHS 140A.
   Color, immature and mature leaves.—Upper surface: RHS 140A. Lower surface: RHS 140B.
   Petioles.—Flat, about 1 mm long by about 0.5 mm wide. The same color as the leaf.
Stem description:
   Length.—About 15 cm.
   Diameter.—About 0.1 cm.
   Internode length.—About 0.7 to 2.0 cm.
   Color.—RHS 142A.
   Texture.—Smooth.
   Stem anthocyanin.—Absent.
   Peduncle.—Color: RHS 142A. Length: About 2.0 to 6.0 cm. Diameter: About 0.07 cm. Texture: Smooth.
Flower description:
   Flower arrangement and form.—Flowers form at apical axils, one flower per axil.
   Natural flowering season, blooming habit.—Continuous.
   Flower longevity on the plant.—About 2 weeks.
   Quantity of flowers and buds per flowering branch.—About 8 to 12.
   Fragrance.—None detected.
   Inflorescence type.—Raceme.
   Flower buds.—Color, when first opening: RHS 118B. Length: About 0.4 cm. Diameter: About 0.2 cm. Shape: Ovate.
   Flower diameter (horizontal).—About 1.2 cm.
   Flower height (vertical).—About 0.8 cm.
   Flower.—Upper lobe: Color: Upper surface: RHS 120B. Lower surface: RHS 118B. Length: About 0.5 to 0.6 cm. Width: About 0.1 cm. Shape: Obovate. Apex: Obtuse margin. Texture: Smooth. — Lower lobe: Color: Upper surface: RHS 120B. Lower surface: RHS 118B. Length: About 0.5 to 0.6 cm. Width: About 0.15 cm. Shape: Obovate. Apex: Obtuse margin. Texture: Smooth.
   Corolla.—Color inside: RHS 119D. Color outside: RHS 118D. Tube length: About 0.3 cm. Diameter, at top of corolla tube: About 0.2 cm.
   Color.—RHS 142A.
   Calyx.—Quantity of sepals: 5 Color: RHS 142A.
   Reproductive organs.—Androecium: Absent. Gynoecium: Pistil length: About 0.6 cm. Stigma color: RHS 92A.
Seeds and fruits: Seed and fruit development have not been observed.
Cultural characteristics:
   Disease and insect resistance.—Typical of species. Suffer from whitefly under Greenhouse conditions.
   Temperature tolerance.—0° C. to about 35° C.
   Frost tolerance.—Will manage light frost but will burn under heavy frost.
   Water tolerance.—Soil needs to be kept damp.

We claim:

1. A new and distinct variety of Lobelia plant (Lobelia pedunculata) named ‘ALMANDA BLUE’, as illustrated and described herein.
